(19) United States
(12) Patent Application Publication
(10) Pub. No.: US 2011/0008846 A1
(43) Pub. Date: Jan. 13, 2011

(54) MODIFIED ENZYMES AND THEIR USES

(75) Inventors: Lars-Erik Peters, Lafayette, CO (US); Nan Fang, Neuss (DE); Janina Cramer, Hilden (DE)

Correspondence Address: Ballard Spahr LLP, SUITE 1000, 999 PEACHTREE STREET, ATLANTA, GA, 30309-3915, US

(73) Assignee: QIAGEN GMBH, Hilden (DE)

(21) Appl. No.: 12/810,714

(22) PCT Filed: Dec. 12, 2008
(86) PCT No.: PCT/EP2008/067440

§ 371 Date: Sep. 28, 2010

(30) Foreign Application Priority Data
Dec. 28, 2007 (EP) 07150478.1

(51) Int. Cl. C12P 19/34; C12N 9/96

(52) U.S. Cl. 435/91.2; 435/188

(57) ABSTRACT

The invention relates to an active enzyme of bacterial, fungal, viral, or archae origin, wherein the enzyme is coupled, preferably covalently coupled with at least one polymer having a molecular weight between about 500 to about 20,000 daltons selected from the group consisting of polyethylene glycol and polypropylene glycol. The invention relates in particular to uses thereof of such as in molecular biology as well as kits comprising such enzymes. In preferred embodiments the enzymes of the invention are nucleic acid modifying or replicating enzymes.

### FIELD OF THE INVENTION

The invention is in the field of molecular biology, diagnostics, and in in-vitro assays. In particular the invention is in the field of enzyme chemistry, more in particular the field of storage and activity of enzymes in particular DNA modifying and amplification enzymes.

### BACKGROUND OF THE INVENTION

Enzymes are proteins that catalyze chemical reactions. Almost all processes in a biological cell need enzymes in order to occur at significant rates. Like all catalysts, enzymes work by lowering the activation energy for a reaction, thus dramatically accelerating the rate of the reaction. As with all catalysts, enzymes are not consumed by the reactions they catalyze, nor do they alter the equilibrium of these reactions. However, enzymes do differ from most other catalysts by being much more specific. Enzymes are known to catalyze about 4,000 biochemical reactions.

Enzyme activity can be affected by other molecules. Inhibitors are molecules that decrease enzyme activity; activators are molecules that increase activity. Many drugs and poisons are enzyme inhibitors. Activity is also affected by temperature, chemical environment (e.g. pH), and the concentration of substrate. Many enzymes are used commercially, for example, in the synthesis of antibiotics. In addition, some household products use enzymes to speed up biochemical reactions (e.g., enzymes in biological washing powders break down protein or fat stains on clothes; enzymes in meat tenderizers break down proteins, making the meat easier to chew).

The stability of peptide bonds ensures that the primary structure of proteins remain intact under biological conditions. Of course, proteases in the environment can cleave the bonds and break a protein apart. Long term chemical changes to proteins include deamidation of asparagine (alkaline pH and phosphates in buffers promote this), oxidation of thiol- or aromatic ring-containing amino acids (usually by heavy metal ions), beta elimination, etc.

The tertiary structures of proteins which largely determine the protein's function are vulnerable to changes in their environment. The chances of adversely affecting proteins increase as they are manipulated more (purification, freezing, thawing). Proteins at 4° C. usually have shelf-lives of only months.

The first hint that a protein has stability problems is the appearance of precipitates in the protein solution. Such precipitation can occur over long term even if precautions have been taken to avoid acute stresses (like freezing and thawing).

One can wear gloves and use clean tubes and pipette tips, protect protein solutions from contamination with proteases and chemicals in the environment.

Proteins need to be stored at a high level of concentration—at least 1 mg/ml. This may not be realistic in some cases and so, if possible, an inert protein such as BSA should be added to raise the total protein concentration to 10-15 mg/ml. The draw back of course is that BSA may interfere with some reactions and applications.

Protein solutions should not be vigorously shaken by vortexing. That not only generates bubbles and raises oxygen levels in the solution, but can also affect protein structure because of the high shearing forces. Many proteins so far require detergents for storage. Shaking such a solution will result in bubble production.

The range of temperatures that a protein goes through during freezing and thawing exposes it to extremes of pH and salt. To lower this degrading effect during freezing, protein solutions should be snap frozen by immersing tubes in dry ice and ethanol/acetone mixture.

One needs to be careful about the buffer used to store a protein in. For enzymes, storage buffers ideally are the same ones in which the enzyme shows good activity. Similarly for pH although it may need to be different if the optimal pH is unsuitable for downstream applications like chromatography. Phosphates in some buffers can inhibit certain enzymes. Some proteins require certain ions like magnesium and/or certain concentration of NaCl for optimal stability. At the same time even 150 mM NaCl can promote aggregation of certain proteins. Reducing environment, because of low pH and reducing agents is usually detrimental to proteins that have disulfide bonds (usually lumenal and extracellular proteins). However, they are usually needed (1-5 mM) for proteins that have free thiol groups, usually intracellular proteins. Some proteins can also be more stable when their ligands are present in the solution. E.g., galectin proteins retain functionality when stored with lactose.

Heavy metal ions can be damaging to many proteins, primarily by oxidizing thiol groups. Reducing agents like DTT and/or 0.1 mM EDTA can help in preventing such damage.

Lyophilization is a useful process for long term storage. However, the protein sample needs to be rapidly frozen before lyophilizing.

Some studies have indicated that 50 mM each of arginine and glutamic acid in solution significantly increases protein stability (and solubility) without adversely affecting protein activity.

Azide (sodium) in the storage solution or thiomerosal can prevent microbial contamination of the solution which is important for 4° C. storage.

Protein solutions can also be stored as ‘salted-out’ with ammonium sulfate (usually 70% saturated). Even at 4° C., such salt-outs are stable for many months. The salt can be removed by dialysis.

Citrate, tris and histidine buffers are less likely to undergo pH changes during freezing and thawing. Sodium phosphate buffers however undergo big changes.

Numerous enzyme applications require certain conditions which are not suited for enzyme activity or storage. In DNA amplification for example there are a number of applications that require a detergent-free reaction buffer system. PCR products used for DHPLC analysis are recommended to be free of detergents. Detergents can cause foaming when PCR products are spotted on microarray slides. However, said detergents have a beneficial effect on polymerase activity and storage. Hence, leaving the detergent out of the storage buffer or the reaction buffer is also not an ideal solution.

There is a need for alternative means for storing and/or reacting isolated enzymes of bacterial, viral, or archae origin.

The prior art knows PEGylation. This is the act of covalently coupling a polyethylene glycol (PEG) and polyethylene oxide (PEO) structure to another larger molecule, for example, a therapeutic protein (which is then referred to as PEGylated). PEGylated interferon alfa-2a or -2b is a commonly used as injectable treatment for Hepatitis C infection.

PEGylation is a process of covalently attaching the strands of the polymer PEG to molecules, most typically peptides, proteins, and antibody fragments, that can help to meet the challenges of improving the safety and efficiency of many therapeutics. It produces alterations in the physiochemical properties including changes in conformation, electrostatic binding, hydrophobicity etc. These physical and chemical changes increase systemic retention of the therapeutic agent. Also, it can influence the binding affinity of the therapeutic moiety to the cell receptors and can alter the absorption and distribution patterns.

PEGylation, by increasing the molecular weight of a molecule, can impart several significant pharmacological advantages over the unmodified form, such as: improved drug solubility, reduced dosage frequency, without diminished efficacy with potentially reduced toxicity, extended circulating life or increased drug stability. U.S. Pat. No. 4,179,337 relates to such applications.

However, so far industrially used enzymes, in particular such enzymes which may modify or replicate a nucleic acid have not been PEGylated.

### SUMMARY OF THE INVENTION

The inventors have astonishingly found that certain industrially used enzymes may be modified by coupling a polymer and that enzymes modified in such a way have beneficial features which are not expected from the prior art. One advantage of such a modification is that enzymes need not be used and/or stored in detergents but retain and/or exhibit activity also in the absence of said detergents.

The invention relates to an isolated, active enzyme of bacterial, fungal, viral, or archae origin, wherein the enzyme is coupled with at least one polymer having a molecular weight between about 500 to about 20,000 daltons selected from the group consisting of polyethylene glycol and polypropylene glycol.

Herein “bacterial, fungal, viral, or archae origin” means that the enzyme may be isolated from said organisms. However, the invention also encompasses such enzymes which are recombinantly produced and/or modified. Modifications include but are not limited to such amino acid changes which enhance the binding of the polymer according to the invention. Modifications include changes of the amino acid sequence which otherwise influence or enhance the activity of the enzyme.

The enzymes according to the invention may be used in industrial methods or in-vitro applications such as in vitro diagnostics.

### DETAILED DESCRIPTION OF THE INVENTION

The invention relates to an active enzyme of bacterial, fungal, viral, or archae origin, wherein the enzyme is coupled with at least one polymer having a molecular weight between about 500 to about 20,000 daltons selected from the group consisting of polyethylene glycol and polypropylene glycol.

The enzymes according to the invention (see some example enzymes in Table 2) may be used in industrial methods or in in-vitro applications a number of which are shown in Table 1.

TABLE 1

| Application | Enzymes used | Uses |
|---|---|---|
| Baking industry | Fungal alpha-amylase enzymes are | Catalyze breakdown of starch in the |
| | normally inactivated at about 50 | flour to sugar. Yeast action on sugar |
| | degrees Celsius, but are destroyed | produces carbon dioxide. Used in |
| | during the baking process. | production of white bread, buns, and |
| | | rolls. |
| | Proteases | Lowers the protein level of flour. |
| Baby foods | Trypsin | Predigestion of baby food |
| Brewing industry | Enzymes from barley are released | Degraded starch and proteins to |
| | during the mashing stage of beer | product simple sugars. |
| | production. | |
| | Industrially produced barley enzymes | Widely used in the brewing process to |
| | | substitute for the natural enzymes |
| | | found in barley. |
| | Amylase, glucanases, proteases | Splits polysaccharides and proteins in |
| | | the malt. |
| | Betaglucanases and arabinoxylanases | Improve the beer filtration |
| | | characteristics. |
| | Amyloglucosidase and pullulanases | Used for low-calorie beer and |
| | | adjustment of fermentability. |
| | Proteases | Used to remove cloudiness produced |
| | | during storage of beers. |
| | Acetolactatedecarboxylase (ALDC) | Used to avoid the formation of diacetyl |
| Fruit juices | Cellulases, pectinases | Used to clarify fruit juices |
| Dairy industry | Rennin, derived from the stomachs of | Used in the manufacture of cheese, |
| | young ruminant animals (like calves | and to hydrolyze proteins. |
| | and lambs). | |
| | Microbially produced enzyme | Use in the dairy industry. |
| | Lipases | Is implemented during the production |
| | | of Roquefort cheese to enhance the |
| | | ripening of the blue-mould cheese. |
| | Lactases | Break down lactose to glucose and |
| | | galactose. |
| Meat tenderizers | Papain | To soften meat for cooking. |
| Starch industry | Amylases, amyloglucosideases and | Converts starch into glucose and |
| | glucosamylases | various syrups. |
| | Glucose isomerase | Converts glucose into fructose in |
| | | production of high fructose syrups |
| | | from starchy materials. |
| Paper industry | Amylases, xylanases, cellulases and | Degrade starch to lower viscosity, |
| | ligninases | aiding sizing and coating paper. |
| | | Xylanases reduce bleach required for |
| | | decolorising; cellulases smooth fibers, |
| | | enhance water drainage, and promote |
| | | ink removal; lipases reduce pitch and |
| | | lignin-degrading enzymes remove |
| | | lignin to soften paper. |
| Biofuel industry | Cellulases | Used to break down cellulose into |
| | | sugars that can be fermented (see |
| | | cellulosic ethanol). |
| | Ligninases | Use of lignin waste |
| Biological detergent | Primarily proteases, produced in an | Used for presoak conditions and direct |
| | extracellular form from bacteria | liquid applications helping with removal |
| | | of protein stains from clothes. |
| | Amylases | Detergents for machine dish washing |
| | | to remove resistant starch residues. |
| | Lipases | Used to assist in the removal of fatty |
| | | and oily stains. |
| | Cellulases | Used in biological fabric conditioners. |
| Contact lens cleaners | Proteases | To remove proteins on contact lens to |
| | | prevent infections. |
| Rubber industry | Catalase | To generate oxygen from peroxide to |
| | | convert latex into foam rubber. |
| Photographic industry | Protease (ficin) | Dissolve gelatin off scrap film, allowing |
| | | recovery of its silver content. |
| Molecular biology | Restriction enzymes, DNA ligase and | Used to manipulate DNA in genetic |
| | polymerases | engineering, important in |
| | | pharmacology, agriculture and |
| | | medicine. Essential for restriction |
| | | digestion and the polymerase chain |
| | | reaction. Molecular biology is also |
| | | important in forensic science. |

Table 1 shows a selection of enzymes used in industry and molecular biology according to the invention.

Polyethylene glycol (PEG) and polyethylene oxide (PEO) are polymers composed of repeating subunits of identical structure, called monomers, and are the most commercially important polyethers. PEG or PEO refers to an oligomer or polymer of ethylene oxide. The two names are chemically synonymous, but historically PEG has tended to refer to shorter polymers, PEO to longer. Both are prepared by polymerization of ethylene oxide.

The numbers that are often included in the names of PEGs and PEOs indicate their average molecular weights, e.g. a PEG with n=80 would have an average molecular weight of approximately 3,500 daltons and would be labeled PEG 3,500. Most PEGs and PEOs include molecules with a distribution of molecular weights, i.e. they are polydisperse. The size distribution can be characterized statistically by its weight average molecular weight (Mw) and its number average molecular weight (Mn), the ratio of which is called the polydispersity index (Mw/Mn). Mw and Mn can be measured by mass spectroscopy.

In a preferred embodiment the enzyme carries at least a nucleic acid modifying or replicating activity.

Such enzymes are for example, nucleases and ligases. Nucleases cut DNA strands by catalyzing the hydrolysis of the phosphodiester bonds. Nucleases that hydrolyse nucleotides from the ends of DNA strands are called exonucleases, while endonucleases cut within strands. The most frequently-used nucleases are the restriction endonucleases, which cut DNA at specific sequences.

Enzymes called DNA ligases can rejoin cut or broken DNA strands, using the energy from either adenosine triphosphate or nicotinamide adenine dinucleotide.

The enzymes according to the invention include topoisomerases and helicases. Topoisomerases are enzymes with both nuclease and ligase activity. These proteins change the amount of supercoiling in DNA. Some of these enzymes work by cutting the DNA helix and allowing one section to rotate, thereby reducing its level of supercoiling; the enzyme then seals the DNA break. Other types of these enzymes are capable of cutting one DNA helix and then passing a second strand of DNA through this break, before rejoining the helix. Helicases use the chemical energy in nucleoside triphosphates, predominantly ATP, to break hydrogen bonds between bases and unwind the DNA double helix into single strands.

Polymerases are particularly preferred according to the invention. Polymerases are enzymes that synthesise polynucleotide chains from nucleoside triphosphates. They function by adding nucleotides onto the 3′ hydroxyl group of the previous nucleotide in the DNA strand. Polymerases are classified according to the type of template that they use.

In DNA replication, a DNA-dependent DNA polymerase makes a DNA copy of a DNA sequence. Accuracy is vital in this process, so many of these polymerases have a proofreading activity. Here, the polymerase recognizes the occasional mistakes in the synthesis reaction by the lack of base pairing between the mismatched nucleotides. If a mismatch is detected, a 3′ to 5′ exonuclease activity is activated and the incorrect base removed. RNA-dependent DNA polymerases are a specialised class of polymerases that copy the sequence of an RNA strand into DNA. They include reverse transcriptase. Transcription is carried out by a DNA-dependent RNA polymerase that copies the sequence of a DNA strand into RNA.

Thus, in one embodiment of the invention such an activity may be selected from the group comprising enzymes with exonuclease activity, enzymes with endonuclease activity, enzymes with polymerising activity, enzymes with methyltransferase activity, enzymes with recombinase activity, enzymes with polynucleotide kinase activity, enzymes with phosphatase activity and enzymes with sulfurylase activity.

The invention relates to an enzyme of bacterial, fungal, viral, or archae origin, wherein the enzyme is covalently coupled with at least one polymer having a molecular weight between about 500 to about 50,000 daltons selected from the group consisting of polyethylene glycol and polypropylene glycol, wherein the enzyme carries at least a nucleic acid modifying and/or replicating activity, wherein the polymer is bound either to an amino acid that is present in the amino acid sequence of the enzyme in its natural state or to an amino acid that has been incorporated into the enzyme either in addition to an existing amino acid or at the position of an existing amino acid. Covalent coupling requires at least one reactive moiety of polyethylene glycol and polypropylene glycol, such as a functional endgroup.

It is preferred that the polymer has a molecular weight between about 750 and 10,000 daltons. When pegylating polymerases it is preferred that the polymer has a molecular weight of between about 2,000 and 8,000 daltons.

It is preferred that the enzyme carries between 1 and 100 polymer moieties per enzyme molecule.

It is preferred that the polymer is polyethylene glycol.

According to the invention it is required that the polymer has at least one functional endgroup, or one homobifunctional endgroup or one heterobifunctional endgroup.

It is further preferred that the polymer has at least one heterobifunctional endgroup selected from the group of maleimide, vinyl sulphones, pyridyl disulphide, amine, carboxylic acids and NHS esters. Maleimide is most preferred.

It is preferred that the polymer is bound to a reactive amino acid selected from the group of lysine, cysteine, histidine, arginine, aspartic acid, glutamic acid, serine, threonine and tyrosine.

It is particularly preferred that the polymer is bound to a reactive amino acid located close to the N-terminal end of the enzyme or close to the C-terminal end of the enzyme.

It is preferred that the polymer is bound to the N-terminal amino group and/or the C-terminal carboxylic acid. This is advantageous because often such binding reduces any negative influence on enzyme activity.

In a preferred embodiment the enzyme is selected from the group of fungal alpha-amylase, protease, trypsin, amylase, glucanase, protease, betaglucanase, arabinoxylanases, amyloglucosidase, pullulanases, proteases, acetolactatedecarboxylase (ALDC), pectinases, rennin, lipases, lactases, papain, glucoamylases, glucose isomerase, xylanase, cellulase, ligninase, restriction enzymes, DNA ligase, polymerase, a ligase, an endonuclease, an exonuclease, methyltransferase, recombinase, polynucleotide kinase, phosphatases sulfurylases.

In a preferred embodiment the enzyme is an enzyme which has a nucleic acid as a substrate.

In one aspect of the invention the enzyme is an E. coli Uracil-N-Glycosylase.

In another aspect of the invention the enzyme is a polymerase.

It is most preferred that the polymerase is a DNA-dependent DNA polymerase, an RNA-dependent DNA polymerase or an RNA-dependent RNA polymerase. Table II discloses preferred enzymes as well as enzymes which have been altered according to the invention.

SEQ ID NO. 1 discloses the wildtype Taq DNA sequence, SEQ ID NO. 2 the corresponding amino acid sequence. This protein may be coupled to a polymer according to the invention. Alternatively, the protein may be modified by incorporating amino acids that serve in binding the polymer of the invention. This has been done for Thermus eggertssonii (SEQ ID NOs. 5 to 8).

TABLE II

| | |
|---|---|
| SEQ ID NO. 1 | Thermus aquaticus—complete nucleotide sequence |
| | of the polA gene-(D3201 |
| SEQ ID NO. 2 | Thermus aquaticus—complete amino acid sequence |
| | of the DNA polymerase I-(D32013) |
| SEQ ID NO. 3 | Thermus eggertssonii wild type—complete |
| | nucleotide sequence of the polA gene |
| SEQ ID NO. 4 | Thermus eggertssonii wild type—complete amino |
| | acid sequence of the DNA polymerase I |
| SEQ ID NO. 5 | Thermus eggertssonii mutant G834C—complete |
| | nucleotide sequence |
| SEQ ID NO. 6 | Thermus eggertssonii mutant G834C—complete |
| | amino acid sequence |
| SEQ ID NO. 7 | Thermus eggertssonii mutant I825C—complete |
| | nucleotide sequence |
| SEQ ID NO. 8 | Thermus eggertssonii mutant I825C—complete |
| | amino acid sequence |
| SEQ ID NO. 9 | E. coli Uracil-N-Glycosylase—complete nucleotide |
| | sequence |
| SEQ ID NO. 10 | E. coli Uracil-N-Glycosylase—complete amino acid |
| | sequence |

It is further preferred that the enzyme is from a thermophilic organism. If the enzyme is a polymerase it is particularly preferred that the polymerase is from a thermophilic organism.

It is preferred that the polymerase according to the invention is selected from the group of genera of Thermus, Aquifex, Thermotoga, Thermocridis, Hydrogenobacter, Thermosynchecoccus and Thermoanaerobacter.

It is preferred that the polymerase according to the invention is selected from the group of organisms of Aquifex aeolicus, Aquifex pyogenes, Thermus thermophilus, Thermus aquaticus, Thermotoga neapofitana, Thermus pacificus and Thermotoga maritima.

DNA polymerases can be subdivided into seven different families: A, B, C, D, X, Y, and RT. For example, prokaryotic DNA polymerase I is an family A polymerase that mediates the process of DNA repair, while polymerase III is the primary enzyme involved with bacterial DNA replication. Discovered by Arthur Kornberg in 1956, DNA polymerase I was the first known DNA polymerase, and was initially characterized in E. coli, although it is ubiquitous in prokaryotes. It is often referred to as simply Pol I. Pol I and its derivatives, such as kienow fragment from E. coli Pol I and thermos aquticus Pol I, are widely used in the molecular biology research. In the present invention it is preferred that the enzyme is a pol-A type polymerase.

It is preferred that the enzyme according to the invention is encoded by a nucleic acid according to SEQ ID NO. 1 (Taq) or shares over 80%, over 85%, over 90% over 95° %, or most preferentially over 98% sequence identity with SEQ ID NO. 1.

It is preferred that the enzyme according to the invention has an amino acid sequence according to SEQ ID NO. 2 (Taq) or shares over 85%, over 90% over 95%, or most preferentially over 98% sequence identity with SEQ ID NO. 2.

It is preferred that the enzyme according to the invention is encoded by a nucleic acid according to SEQ ID NO. 3 (Teg) or shares over 80%, over 85%, over 90% over 95° %, or most preferentially over 98% sequence identity with SEQ ID NO. 3.

It is preferred that the enzyme according to the invention has an amino acid sequence according to SEQ ID NO. 4 (Teg) or shares over 85%, over 90% over 95%, or most preferentially over 98% sequence identity with SEQ ID NO. 4.

It is preferred that the enzyme according to the invention is encoded by a nucleic add according to SEQ ID NO. 5 (Thermus eggertssonii mutant G834C) or shares over 80%, over 85%, over 90% over 95%, or most preferentially over 98% sequence identity with SEQ ID NO. 5.

It is preferred that the enzyme according to the invention has an amino acid sequence according to SEQ ID NO. 6 (Thermus eggertssonii mutant G834C) or shares over 85%, over 90% over 95%, or most preferentially over 98% sequence identity with SEQ ID NO. 6.

It is preferred that the enzyme according to the invention is encoded by a nucleic acid according to SEQ ID NO. 7 (Thermus eggertssonii mutant 18250) or shares over 80%, over 85%, over 90% over 95%, or over most preferentially over 98% sequence identity with SEQ ID NO. 7.

It is preferred that the enzyme according to the invention has an amino acid sequence according to SEQ ID NO. 8 (Thermus eggertssoni mutant 18250) or shares over 85%, over 90% over 95%, or most preferentially over 98% sequence identity with SEQ ID NO. 8.

The polymerases according to the invention which are modified by the addition of the polymer according to the invention may have also other modifications. In one embodiment, the variant Teg DNA polymerase I (which carries a polymer) comprises an amino acid sequence having a substitution at position 679 of SEQ ID NO. 4 replacing the glutamic acid residue by a positively charged amino acid such as lysine or arginine. Analysis of the three dimensional structure of Taq DNA polymerase I bound to a DNA substrate has shown that the negative charge of the glutamic acid at the corresponding position (681) in the Taq DNA polymerase sequence (SEQ ID NO.° 2) contacts the negatively charged phosphate backbone of the priming strand in the DNA substrate. That contact creates an electrostatic repulsion effect limiting the extension rate and processivity of the polymerase. Mutant variants carrying a lysine instead of glutamic acid at the position have shown faster extension rates and better processivity. Variant Teg DNA polymerases with those features are desirable for various applications, such as fast PCR, DNA sequencing, amplification of long target sequences. The invention also relates to other pol-A type polymerases with said mutation. Upon alignment the person skilled in the art will identify the respective amino acid.

The polymerases according to the invention which are modified by the addition of the polymer according to the invention may have also further modifications. In one embodiment, the variant Teg DNA polymerase I comprises an amino acid sequence having a substitution at position 683 of SEQ ID NO. 4 replacing the glutamic acid residue by a positively charged amino acid such as lysine or arginine. Analysis of the three dimensional structure of Taq DNA polymerase I bound to a DNA substrate has shown that the negative charge of the glutamic acid at the corresponding position (681) in the Taq DNA polymerase sequence (SEQ ID NO. 2) contacts the negatively-charged phosphate backbone of the priming strand in the DNA substrate. That contact creates an electrostatic repulsion effect limiting the extension rate and processivity of the polymerase. Mutant variants carrying a lysine instead of glutamic acid at the position have shown faster extension rates and better processivity. Variant Teg DNA polymerases with those features are desirable for various applications, such as fast PCR, DNA sequencing, amplification of long target sequences.

The polymerases according to the invention which are modified by the addition of the polymer according to the invention may have also further modifications. In one embodiment, a variant Teg DNA polymerase I comprises an amino acid sequence having single or combined substitutions at the positions 612-613 of SEQ ID NO. 4. In one embodiment, a variant Teg DNA polymerase I comprises an amino acid sequence having single or combined substitutions at the positions 616-617 of SEQ ID NO. 4. Random mutagenesis experiments performed on Taq and E. coli DNA polymerase I have shown that the amino acid residues at the corresponding positions in their sequence control discrimination between rNTPs and dNTPs as polymerization substrate. They also control discrimination between RNA- or DNA-primed DNA templates, templates with base mismatches at the 3′-terminus of the primer and perfectly annealed primers and between labeled and non-labeled dNTP substrates. Based on the nature of the substitution(s) at these positions, a number of variant Teg DNA Pol I can be provided with useful features for different applications. Variants with increased discrimination against the extension of mismatched primers are useful for allele-specific PCR. Variants with increased affinity for labeled dNTP substrates are useful for fluorescent DNA sequencing and real-time PCR.

The inventors have found that certain enzymes which have been adapted for better pegylation have very good properties. The invention thus also relates to a polymerase encoded by a nucleic acid according to SEQ ID NO. 5 or SEQ ID NO. 7. The invention also relates to a polymerase with an amino acid sequence according to SEQ ID NO. 6 and SEQ ID NO. 8.

In another embodiment of the invention, the variant of Teg DNA Pol 1 is based on the knowledge that a single residue in DNA polymerases of Thermus aquaticus DNA polymerase I family is critical for distinguishing between deoxy- and dideoxyribonucleotides (Taber, S., Richardson, C. C., Proc. Natl. Acad. Sci. USA, 1995, July 3, 92 (14): 6339-43, A single residue and DNA polymerase of the Escherichia coli DNA polymerase I family is critical for distinguishing between deoxy- and dideoxyriboncleotides). In a preferred embodiment, any Pol I variant according to the invention comprises an amino acid sequence having a substitution residue in place of a wildtype phenylalanine in a position corresponding to position 665 of SEQ ID NO. 2. In a preferred embodiment, the substitution residue is a tyrosine. In a preferred embodiment, the Pol I variant comprises an amino acid sequence having a substitution residue in place of a wildtype phenylalanine in a position corresponding to position 669 of Taq. In a preferred embodiment, the substitution residue is a tyrosine.

In one embodiment, the variant Teg DNA Pol I which carries a polymer has four additional amino acid residues Met, Pro, Arg/Lys and Gly at the N-terminus of the amino acid sequence set forth in SEQ ID NO. 4. Based on the deciphered three dimensional structure of Taq DNA polymerase bound to DNA substrate these three additional N-terminal residues are a part of the DNA-binding site in the N-terminal nuclease domain. In the absence of the additional N-terminal amino acids the Teg DNA polymerase has a weakened binding affinity and strength towards its DNA substrate. Teg DNA Pol I variants with strengthened DNA substrate binding properties have better processivity and a faster extension rate than Teg DNA Pol I with the wild-type sequence. Improved processivity and faster extension rates are important functional features of thermostable DNA polymerases used to perform the polymerase chain reaction (PCR) application. They allow for amplification of longer target sequences with higher sensitivity requiring less DNA template in the sample. The additional praline residue in position 2 of the variant Teg DNA Pol I in this embodiment stabilizes the recombinant polymerase against N-terminal degradation by endogenous cytoplasmic proteinases of the E. coli host cells according to the rules of stabilizing N-terminal amino acid residues in E. coli well established in the prior art.

The enzyme according to the invention may be a fusion protein. Teg DNA Pol I proteins of the invention also include DNA Pol I fusion proteins that comprise a Teg DNA Pol I protein fused to a non-Teg DNA Pol I protein moiety. In one embodiment, a DNA Pol I fusion protein comprises an exonuclease domain of a Teg DNA Pol I protein of the invention. In one embodiment, a DNA Pol I fusion protein comprises a polymerase domain of a Teg DNA Pol I protein of the invention. DNA Pol I fusion proteins of the invention may include moieties that, for example, provide for purification, or contribute to the altered thermostability or altered catalytic activity of a DNA Pol I fusion protein as compared to a Teg DNA Pol I protein.

It is preferred that at least one polymer is coupled to an amino acid that is not present in the amino acid sequence of the enzyme in its natural state but has been incorporated into the enzyme either in addition to an existing amino acid or at the position of an existing amino acid.

It is preferred that at least one polymer is coupled to an amino acid selected from the group of lysine and cysteine.

The amino acid is ideally surface exposed according to the invention.

Polymerase according to the invention, wherein the at least one polymer is coupled to one or more cysteine and/or lysine residues that have been incorporated into the polymerase at a position corresponding to the position of the following amino acids of Taq polymerase Leu⁴⁶¹, Ala⁵²¹, Gly⁶⁴⁸, Ala⁶⁵³, Ser⁶⁷⁹, Ala⁶⁸³, Ser⁶⁹⁹, Ser⁷³⁹ Ala⁸¹⁴, Ser⁸²⁹ and Glu⁸³². The complete Taq amino acid sequence is shown in SEQ ID NO. 2.

The invention also relates to methods for modifying or amplifying a nucleic acid,

wherein the nucleic acid to be modified is present in a reaction mixture comprising an enzyme according to the invention. The methods comprise subjecting a nucleic acid molecule to a reaction mixture comprising an enzyme of the invention. Preferably the method is an amplification reaction and the enzyme is a polymerase.

In a preferred embodiment, the nucleic acid molecule used in the amplification method is DNA. In a preferred embodiment, the DNA molecule is double stranded. In other embodiments, the DNA molecule is single stranded. In a preferred embodiment, the double stranded DNA molecule is a linear DNA molecule. In other embodiments, the DNA molecule is non-linear, for example circular or supercoiled DNA.

In a preferred embodiment, the amplification method is a thermocycling amplification method useful for amplifying a nucleic acid molecule, preferably DNA, which is preferably double stranded, by a temperature-cycled mode. In a preferred embodiment, the method involves subjecting the nucleic acid molecule to a thermocycling amplification reaction in a thermocycling amplification reaction mixture. The thermocycling amplification reaction mixture comprises a Teg DNA Pol I protein of the invention.

In a preferred embodiment, the amplification method is a PCR method. In one embodiment, the method is a degenerate PCR method. In one embodiment, the method is a real-time PCR method.

In one embodiment, the invention provides reaction mixtures for nucleic acid amplification, which comprise a Teg DNA Pol I protein or a polymerase which are modified with the polymer of the invention. Preferred reaction mixtures of the invention are useful for DNA amplification. In a preferred embodiment, the reaction mixture is a thermocycling reaction mixture useful for thermocycling amplification reactions. Amplification reaction mixtures may include additional reagents, such as, but not limited to, dNTPs, primers, buffer, and/or stabilizers.

In one embodiment, the invention provides reaction mixtures for amplifying nucleic acids using degenerate primers in PCR, which are useful for the amplification of homologous sequence targets containing nucleotide polymorphisms. The reaction mixtures comprise a Teg DNA Pol I protein or a polymerase which is modified with the polymer of the invention. Reaction mixtures for PCR with degenerate primers may include additional reagents such as, but not limited to, dNTPs, degenerate primers, buffer, and/or stabilizers.

In a preferred embodiment, the reaction mixture comprises a Teg DNA Pol I protein of the invention, wherein the Teg DNA Pol I is present in the reaction mixture at a concentration of not less than 120 pg/μL, more preferably not less than 140 pg/μL, more preferably not less than 160 pg/μL, more preferably not less than 180 pg/μL, more preferably not less than 200 pg/μL, more preferably not less than 400 pg/μL, more preferably not less than 600 pg/μL.

In a preferred embodiment, the reaction mixture comprises a zwitterionic buffer. In a preferred embodiment, the zwitterionic buffer has a pH between about pH 7.5-8.9. In a preferred embodiment, the buffer comprises a combination of an organic zwitterionic acid and an organic zwitterionic base, potassium ions, and magnesium ions. In an especially preferred embodiment, the reaction mixture comprises 30 mM Bicine, 59 mM Tris, 50 mM KCl, 2 mM magnesium acetate.

In one embodiment, the invention provides reaction mixtures for amplifying nucleic acids, which are useful in PCR reactions with real time product detection. The real-time reaction mixtures comprise a polymerase of the invention preferably a Teg DNA Pol I of the invention. The real-time PCR reaction mixtures may include other reagents, including, but not limited to, dNTPs, fluorescent probes, primers, buffer, stabilizers, nucleic acid-binding dye(s) and/or passive reference dye(s).

In a preferred embodiment, the reaction mixture comprises a Teg DNA Poi 1, wherein the thermostable Teg Polymerase I is present in the reaction mixture at a concentration of not less than 120 pg/μL, more preferably not less than 140 pg/μL, more preferably not less than 160 pg/μL, more preferably not less than 180 pg/μL, more preferably not less than 200 pg/μL, more preferably not less than 400 pg/μL, more preferably not less than 600 pg/μL.

In a preferred embodiment, the reaction mixture comprises a zwitterionic buffer. In a preferred embodiment, the zwitterionic buffer has a pH between about pH 7.5-8.9. In a preferred embodiment, the buffer comprises a combination of an organic zwitterionic acid and an organic zwitterionic base, potassium ions, and magnesium ions. In an especially preferred embodiment, the reaction mixture comprises a buffer comprising 40 mM Bicine, 90 mM Tris, 40 mM KCl, 4 mM magnesium acetate, and 100 mM sorbitol.

In another preferred embodiment, the reaction mixture comprises a buffer comprising 25 mM Taps, 0.05 mg/mL Anti-freeze Protein I, 10.3 mM Tris, 50 mM KCl, 5 mM magnesium acetate, 100 mM sorbitol, and 0.2 mg/mL BSA.

In one aspect, the invention provides nucleic acid amplification reaction tubes, which comprise a polymerase of the invention preferably a Teg DNA Pol I in a nucleic acid amplification reaction mixture disclosed herein.

In a preferred embodiment, the amplification reaction tubes are thermocycling amplification reaction tubes, which comprise a Teg DNA Pol I in a thermocycling amplification reaction mixture disclosed herein.

In a preferred embodiment, the thermocycling amplification reaction tubes are PCR reaction tubes, which comprise a Teg DNA polymerase I in a PCR reaction mixture disclosed herein.

In a preferred embodiment, the PCR reaction tubes are degenerative PCR reaction tubes, which comprise a Teg DNA Pol I in a degenerative PCR reaction mixture disclosed herein.

In another preferred embodiment, the PCR reaction tubes are real-time PCR reaction tubes, which comprise a Teg DNA Pol I in a real-time PCR reaction mixture disclosed herein.

In one aspect, the invention provides a nucleic acid amplification kit useful for amplifying nucleic acid, preferably DNA, which is preferably double stranded, which comprises a polymerase of the invention preferably a Teg DNA Pol I disclosed herein. In a preferred embodiment, the amplification kit comprises an amplification reaction mixture disclosed herein.

In a preferred embodiment, the amplification kit is a thermocycling amplification kit useful for amplifying nucleic acids, preferably DNA, which is preferably double stranded, by a temperature-cycled mode. The thermocycling amplification kit comprises a Teg DNA Pol I disclosed herein. Preferably, the thermocycling amplification kit comprises a thermocycling amplification reaction mixture disclosed herein.

In a preferred embodiment, the thermocycling amplification kit is a PCR kit for amplifying nucleic acids, preferably DNA, which is preferably double-stranded, by PCR. The PCR kit comprises a polymerase according to the invention preferably a Teg DNA Pol I disclosed herein. Preferably the PCR kit comprises a PCR reaction mixture disclosed herein.

In a preferred embodiment, the PCR kit is a degenerative PCR kit, preferably comprising a degenerative PCR reaction mixture disclosed herein.

In another preferred embodiment, the PCR kit is a real-time PCR kit, preferably comprising a real-time PCR reaction mixture disclosed herein.

In a preferred embodiment, a nucleic acid amplification kit provided herein comprises a nucleic acid amplification reaction mixture, which comprises an amount of a polymerase according to the invention preferably Teg DNA Pol I such that the reaction mixture can be combined with template DNA, primer(s) and/or probe(s) hybridizable thereto, and optionally appropriately diluted to produce a charged reaction mixture, wherein the thermostable DNA Pol I is capable of amplifying the DNA template by extending the hybridized primer(s).

In one embodiment the invention relates to a method of pegylating a nucleic acid modifying enzyme, wherein the enzyme is altered by the introduction of an amino acid or by the alteration of an amino acid, to which polyethylene glycol or polypropylene glycol binds covalently.

Preferably, the altered enzyme has an additional lysine and/or cysteine.

### FIGURE CAPTIONS

FIG. 1

FIG. 1A shows the carboxyl-terminal sequences of the wild type (WT) Teg (from amino acid 776 to 834, as well as the corresponding nucleotide sequence).). FIG. 1 B shows the carboxyl-terminal sequences of the Teg G834C mutant (from amino acid 776 to 834, as well as the corresponding nucleotide sequence; the amino acid 834 was changed from Glycine to Cysteine.).). FIG. 1C shows the carboxyl-terminal sequences of the Teg 1825C mutant (from amino acid 776 to 834, as well as the corresponding nucleotide sequence; the amino acid 825 was changed from Isoleucine to Cysteine). The WT and mutant Teg constructs were cloned in pQE 80 L Kanamycin vector.

FIG. 2

FIG. 2 are protein gel images from Agilent 2100 Bloanalyzer. The increased molecular weights of Teg mutant proteins after pegylation demonstrated that Teg mutants were successfully conjugated with methoxypolyethylene glycol 5,000 maleimide.

FIG. 3

FIG. 3 shows that pegylated Teg DNA polymerase produced more PCR product in the absence of detergents. NC, negative control, which included un-pegylated Teg mutants and free PEG molecules in the PCR reaction; +PEG: PCR with pegylated Teg mutants.

FIG. 4

FIG. 4 shows that Teg polymerase could not generate PCR products in the absence of detergents, even if PEG8000, another PEG isoform, was added directly into the PCR reactions at different concentrations.

### EXAMPLES

### Pegylation of Thermus eggertssonii (Teg) Polymerase Enhances its Performance in PCR in the Absence of Detergent

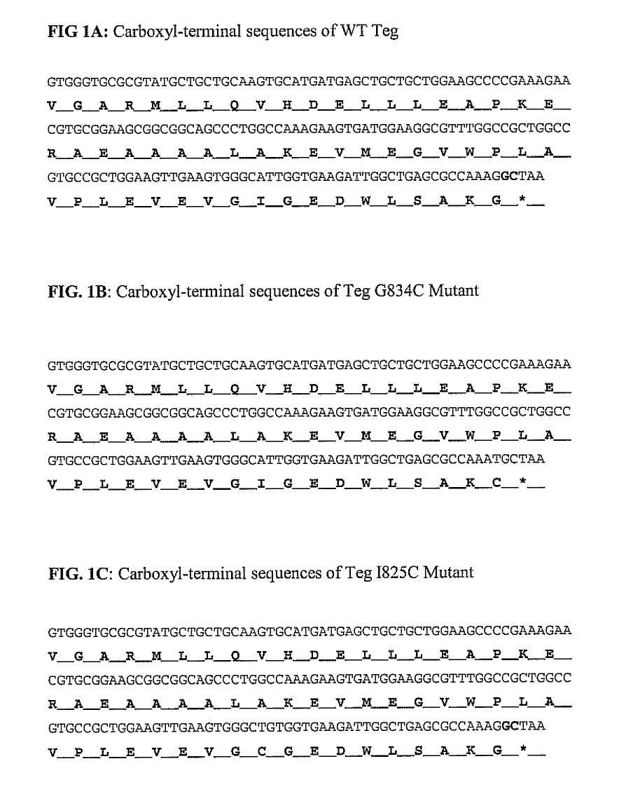

Teg, a thermostable DNA polymerase, does not have any cysteine amino acid in its wildtype form. Therefore, it is only possible to carry out site-specific pegylation with Teg mutants containing cysteine and PEG-maleimide. We used site-directed mutagenesis method (QuikChange XL Site-Directed Mutagenesis Kit, Stratagene) to introduce cysteine on the C-terminus of the protein: 1. Teg 1825C, where the 825th amino acid of Teg, isoleucine, was changed to cysteine; 2. Teg G834C, where the last amino acid glycine of Teg was changed to cysteine. (FIG. 1, wildtype and mutant sequences of the C-terminus of Teg.)

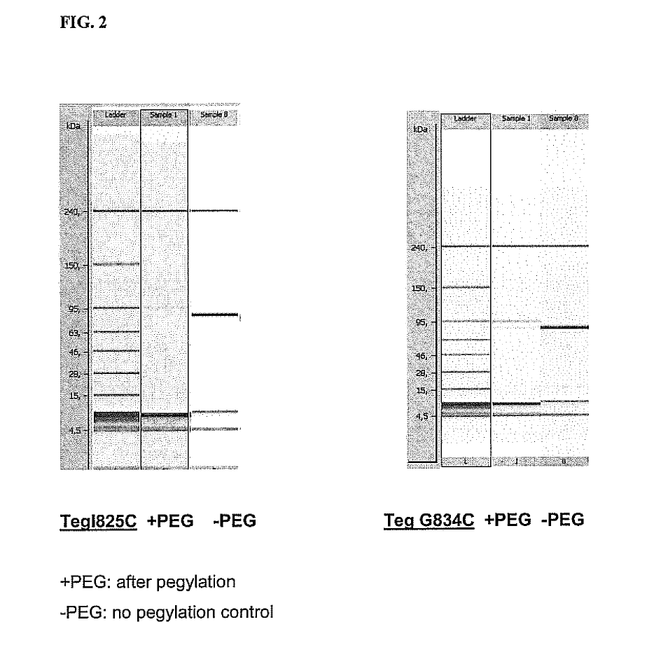

The two Teg mutants, Teg 1825C and Teg G834C, were expressed in E. coli cells, purified, and resuspended in 100 μM HEPES buffer at the concentrations of 6.7 pg/μl and 12 pg/μl, respectively. 10 μl each of Teg 1825C and Teg G834C was mixed with 2.4 μl and 4 μl, respectively, of the 10 mg/ml PEG-maleimide (Methoxypolyethylene glycol 5,000 maleimide, 63187, Fluka/Sigma) to ensure the same molecular ratio of polymerase:PEG for both mutants. The Teg/PEG mixture was diluted with 100 μM HEPES to a final volume of 500 μl and incubated at 4° C. overnight. Successful pegylation (>90% of the protein was pegylated) was demonstrated by the appearance of an additional protein band with lower electrophoresis mobility in the Agilent protein gel (FIG. 2).

Next, we examined the stability of the pegylated Teg mutants in a PCR reaction.

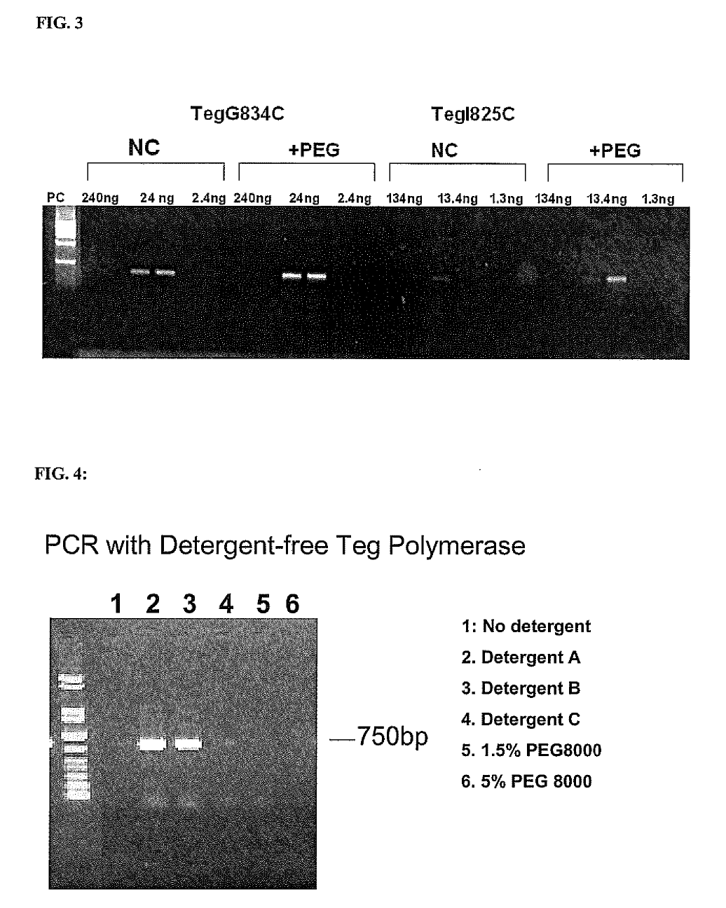

Detergents are normally required to stabilize thermostable polymerase in the PCR (FIGS. 3 and 4). 1°μl from the not diluted, 1:10, or 1:100 diluted pegylation reaction (the amount of the Teg 1825C and Teg G834C was indicated in FIG. 3) was used in 25 μl PCR to amplify a 750-bp amplicon from the human genomic DNA in the absence of detergents.

Negative controls (NC) are unpegylated Teg proteins with PEG-maleimide directly added to the PCR reaction. PCR was performed in duplicates. As shown in FIG. 3, unpegylated Teg generated no or very low level of PCR product in the absence of detergents, even with the free PEG-meleimide in the reaction solutions. This is also confirmed when another form of PEG, PEG8000, was directly added to the detergent-free PCR reactions at different concentrations (FIG. 4). In contrast, pegylated Teg mutants produced higher yield of PCR products compared to negative controls. This demonstrated that Teg polymerase, in the absence of any detergents, can only effectively amplify template with covalently conjugated PEG moleculars.

1. A composition comprising an enzyme covalently coupled with at least one polymer having a molecular weight between about 500 to about 50,000 daltons, wherein the at least one polymer is polyethylene glycol or polypropylene glycol, wherein the enzyme has a nucleic acid modifying or replicating activity, wherein the at least one polymer is bound either to an amino acid that is present in the amino acid sequence of the enzyme in its natural state or to an amino acid that has been incorporated into the enzyme either in addition to an existing amino acid or at the position of an existing amino acid, and wherein the enzyme is of bacterial, fungal, viral, or archae origin.

2. The composition of claim 1, wherein the polymer has a molecular weight of between about 750 and 20,000 daltons.

3. The composition of claim 1, wherein the enzyme carries between 1 and 100 polymer moieties per enzyme molecule.

4. The composition of claim 1, wherein at least one polymer is polyethylene glycol.

5. The composition of claim 1, wherein at least one polymer has at least one functional endgroup, or one homobifunctional endgroup or one heterobifunctional endgroup.

6. The composition of claim 5, wherein at least one polymer has at least one heterobifunctional endgroup selected from the group of maleimide, vinyl sulphones, pyridyl disulphide, amine, carboxylic acids or NHS esters.

7. The composition of claim 6, wherein at least one endgroup is maleimide.

8. The composition of claim 1, wherein polymer is bound to at least one reactive amino acid comprising lysine, cysteine, histidine, arginine, aspartic acid, glutamic acid, serine, threonine or tyrosine.

9. The composition of claim 1, wherein at least one polymer is bound to a reactive amino acid located close to the N-terminal end of the enzyme or close to the C-terminal end of the enzyme.

10. The composition of claim 1, wherein the polymer is bound to the N-terminal amino group of the enzyme and/or the C-terminal carboxylic acid of the enzyme.

11. The composition of claim 1, wherein the enzyme is a polymerase, ligase, endonuclease, exonuclease, methyltransferase, recombinase, polynucleotide kinase, phosphatase or sulfurylase.

12. The composition of claim 11, wherein the polymerase is a DNA-dependent DNA polymerase, an RNA-dependent DNA polymerase, or an RNA-dependent RNA polymerase.

13. The composition of claim 12, wherein the polymerase is from a thermophilic organism.

14. The composition of claim 13, wherein the polymerase is from the genii of Thermus, Aquifex, Thermotoga, Thermocridis, Hydrogenobacter, Thermosynchecoccus or Thermoanaerobacter.

15. The composition of claim 14, wherein the polymerase is from Aquifex aeolicus, Aquifex pyogenes, Thermus thermophilus, Thermus aquaticus, Thermotoga neapolitana, Thermus pacificus or Thermotoga maritima.

16. The composition of claim 12, wherein the enzyme is a Pol-A-type polymerase.

17. The composition of claim 16, wherein the at least one polymer is coupled to lysine, cysteine, or both lysine and cysteine.

18. The composition of claim 12, wherein the amino acid is surface exposed.

19. The composition of claim 16, wherein the at least one polymer is coupled to one or more cysteine and/or lysine residues that have been incorporated into a Pol-A-type polymerase at a position corresponding to the position of the following amino acids of Taq polymerase Leu461, Ala521, Gly648, Ala653, Ser679, Ala683, Ser699, Ser739 Ala814, Ser829 and Glu832.

20. A method of modifying or amplifying a nucleic acid, wherein the nucleic acid to be modified is present in a reaction mixture comprising a polymerase covalently coupled with at least one polymer having a molecular weight between about 500 to about 50,000 daltons wherein at least one polymer is polyethylene glycol or polypropylene glycol or both, wherein the polymerase has at least a nucleic acid modifying and/or replicating activity, wherein at least one polymer is bound either to an amino acid that is present in the amino acid sequence of the polymerase in its natural state or to an amino acid that has been incorporated into the polymerase either in addition to an existing amino acid or at the position of an existing amino acid, and wherein the polymerase is a DNA-dependent DNA polymerase, an RNA-dependent DNA polymerase, or an RNA-dependent RNA polymerase.

21. The method of claim 20, wherein the reaction is an amplification reaction.

22. A method of pegylating a nucleic acid modifying enzyme, wherein the enzyme is altered by the introduction of an amino acid or by the alteration of an amino acid, to which polyethylene glycol or polypropylene glycol may bind covalently.

23. A kit comprising a composition comprising an enzyme covalently coupled with at least one polymer having a molecular weight between about 500 to about 50,000 daltons wherein at least one polymer is polyethylene glycol or polypropylene glycol or both, wherein the enzyme has at least a nucleic acid modifying and/or replicating activity, wherein at least one polymer is bound either to an amino acid that is present in the amino acid sequence of the enzyme in its natural state or to an amino acid that has been incorporated into the enzyme either in addition to an existing amino acid or at the position of an existing amino acid, and wherein the enzyme is of bacterial, fungal, viral, or archae origin.


SEQUENCE LISTING (10 sequences)

SEQ ID NO: 1 - DNA, 2499 bp, Thermus aquaticus
atgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgccctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 2 - protein, 832 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHALHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 3 - DNA, 2503 bp, Thermus eggertssonii
atgggccgtggtatgctgccgctgtttgaaccgaaaggccgtgtgctgctggttgatggccaccatctggcctatcgtaacttttttgcgctgaaaggcctgaccacgagccgtggtgaaccggtgcagggcgtgtatggctttgcgaaaagcctgctgaaagcgctgaaagaggatggcgacgttgttattgtggtgtttgatgcgaaagcgccgttttttcgtcatgaagcgtacgaagcgtataaagcgggccgtgcgccgaccccggaagattttccgcgtcagctggccctgattaaagagctggttgatctgctgggcctggaacgtctggaagtgccgggctttgaagcggatgatgtgctggccaccctggccaaacaggcggaacgtgaaggctatgaagtgcgtattctgaccgcggatcgtgacctgtttcagctgctgagcgatcgtattgcggtgctgcatccggaaggccatctgattacgccgggctggctgtgggaacgttatggcctgaaaccggaacagtgggtggattttcgtgcgctggccggcgatccgagcgataacattccgggcgtgaaaggcattggcgaaaaaaccgcgctgaaactgctgaaagaatggggcagcctggaaaatctgctgaaaaacctggatcatgtgaaaccgccgagcgtgcgtgaaaaaattctggcccatctggatgatctgcgtctgtctcaggagctgtctcgcgttcgtaccgatctgccgctgaaagtggattttaaaaaacgtcgtgaaccggatcgtgaaggcctgaaagcgtttctggaacgcctggaatttggcagcctgctgcatgaatttggcctgctggaaagcccgctgccggcggaagaggcgccgtggccgccaccggaaggtgcgtttctgggctatcgtctgagccgtccggaaccgatgtgggcggagctgctggccctggccgcgagcgcgaaaggtcgtgtgtatcgtgcggaagaaccgtatggcgcgctgcgtggcctgaaagaagtgcgcggcctgctggctaaagacctggccgtgctggccctgcgtgaaggtctggatctgccgccgaccgatgatccgatgctgctggcctatctgctggacccgagcaacaccaccccggaaggtgtggcgcgtcgttatggcggcgaatggaccgaagaagcgggcgaacgcgcggttctgagcgaacgtctgtatgaaaacctgctgggccgtctgcgtggcgaagaaaaactgctgtggctgtatgaagaagtggaaaaaccgctgagccgtgtgctggcccatatggaagcgaccggcgtgcgtctggatgtggcgtatctgaaagccctgagcctggaagtggcggaagaaatgcgtcgtctggaagaagaagtgtttcgtctggccggccatccgtttaacctgaacagccgtgatcagctggaacgtgtgctgtttgatgagctgggcctgccgccgattggcaaaaccgaaaaaaccggcaaacgtagcaccagcgcggcggttctggaagcgctgcgtgaagcgcatccgattgtggaaaaaatcctgcaatatcgtgagctggccaaactgaaaggcacctatattgatccgctgccggccctggtgcatccgaaaaccggccgtctgcatacccgttttaaccagaccgcgaccgcgaccggtcgtctgagcagcagcgatccgaacctgcaaaacattccggtgcgtaccccgctgggccagcgtattcgtcgtgcgtttgtggccgaagaaggctatctgctggttgcgctggattaagccagattgagctgcgtgttctggcccacctgagcggcgatgaaaatctgattcaggtgtttcaggaaggccgcgatattcatacccagaccgcgagctggatgtttggcctgccggccgaagcgatcgatccgctgatgcgtcgtgcggcgaaaaccattaactttggcgtgctgtatggcatgagcgcgcatcgcctgagccaggagctgagcattccgtacgaagaagcggtggcgtttattgatcgttatttccagagctacccgaaagtgaaagcgtggattgaacgtaccctggaagaaggccgtcagcgcggctatgtggaaaccctgtttggccgtcgtcgttatgtgccggatctgaacgcgcgtgtgaaaagcgttcgtgaagcggcggaacgtatggcgtttaacatgccggttcagggcaccgcggcggatctgatgaaactggcaatggtgcgtctgtttccgcgtctgccggaagtgggtgcgcgtatgctgctgcaagtgcatgatgagctgctgctggaagccccgaaagaacgtgcggaagcggcggcagccctggccaaagaagtgatggaggcgtttggccgctggccgtgccgctggaagttgaagtgggcattggtgaagattggctgagcgccaaaggctaa

SEQ ID NO: 4 - protein, 834 aa, Thermus eggertssonii
MGRGMLPLFEPKGRVLLVDGHHLAYRNFFALKGLTTSRGEPVQGVYGFAKSLLKALKEDGDVVIVVFDAKAPFFRHEAYEAYKAGRAPTPEDFPRQLALIKELVDLLGLERLEVPGFEADDVLATLAKQAEREGYEVRILTADRDLFQLLSDRIAVLHPEGHLITPGWLWERYGLKPEQWVDFRALAGDPSDNIPGVKGIGEKTALKLLKEWGSLENLLKNLDHVKPPSVREKILAHLDDLRLSQELSRVRTDLPLKVDFKKRREPDREGLKAFLERLEFGSLLHEFGLLESPLPAEEAPWPPPEGAFLGYRLSRPEPMWAELLALAASAKGRVYRAEEPYGALRGLKEVRGLLAKDLAVLALREGLDLPPTDDPMLLAYLLDPSNTTPEGVARRYGGEWTEEAGERAVLSERLYENLLGRLRGEEKLLWLYEEVEKPLSRVLAHMEATGVRLDVAYLKALSLEVAEEMRRLEEEVFRLAGHPFNLNSRDQLERVLFDELGLPPIGKTEKTGKRSTSAAVLEALREAHPIVEKILQYRELAKLKGTYIDPLPALVHPKTGRLHTRFNQTATATGRLSSSDPNLQNIPVRTPLGQRIRRAFVAEEGYLLVALDYSQIELRVLAHLSGDENLIQVFQEGRDIHTQTASWMFGLPAEAIDPLMRRAAKTINFGVLYGMSAHRLSQELSIPYEEAVAFIDRYFQSYPKVKAWIERTLEEGRQRGYVETLFGRRRYVPDLNARVKSVREAAERMAFNMPVQGTAADLMKLAMVRLFPRLPEVGARMLLQVHDELLLEAPKERAEAAAALAKEVMEGVWPLAVPLEVEVGIGEDWLSAKG

SEQ ID NO: 5 - DNA, 2503 bp, Thermus eggertssonii
atgggccgtggtatgctgccgctgtttgaaccgaaaggccgtgtgctgctggttgatggccaccatctggcctatcgtaacttttttgcgctgaaaggcctgaccacgagccgtggtgaaccggtgcagggcgtgtatggctttgcgaaaagcctgctgaaagcgctgaaagaggatggcgacgttgttattgtggtgtttgatgcgaaagcgccgttttttcgtcatgaagcgtacgaagcgtataaagcgggccgtgcgccgaccccggaagattttccgcgtcagctggccctgattaaagagctggttgatctgctgggcctggaacgtctggaagtgccgggctttgaagcggatgatgtgctggccaccctggccaaacaggcggaacgtgaaggctatgaagtgcgtattctgaccgcggatcgtgacctgtttcagctgctgagcgatcgtattgcggtgctgcatccggaaggccatctgattacgccgggctggctgtgggaacgttatggcctgaaaccggaacagtgggtggattttcgtgcgctggccggcgatccgagcgataacattccgggcgtgaaaggcattggcgaaaaaaccgcgctgaaactgctgaaagaatggggcagcctggaaaatctgctgaaaaacctggatcatgtgaaaccgccgagcgtgcgtgaaaaaattctggcccatctggatgatctgcgtctgtctcaggagctgtctcgcgttcgtaccgatctgccgctgaaagtggattttaaaaaacgtcgtgaaccggatcgtgaaggcctgaaagcgtttctggaacgcctggaatttggcagcctgctgcatgaatttggcctgctggaaagcccgctgccggcggaagaggcgccgtggccgccaccggaaggtgcgtttctgggctatcgtctgagccgtccggaaccgatgtgggcggagctgctggccctggccgcgagcgcgaaaggtcgtgtgtatcgtgcggaagaaccgtatggcgcgctgcgtggcctgaaagaagtgcgcggcctgctggctaaagacctggccgtgctggccctgcgtgaaggtctggatctgccgccgaccgatgatccgatgctgctggcctatctgctggacccgagcaacaccaccccggaaggtgtggcgcgtcgttatggcggcgaatggaccgaagaagcgggcgaacgcgcggttctgagcgaacgtctgtatgaaaacctgctgggccgtctgcgtggcgaagaaaaactgctgtggctgtatgaagaagtggaaaaaccgctgagccgtgtgctggcccatatggaagcgaccggcgtgcgtctggatgtggcgtatctgaaagccctgagcctggaagtggcggaagaaatgcgtcgtctggaagaagaagtgtttcgtctggccggccatccgtttaacctgaacagccgtgatcagctggaacgtgtgctgtttgatgagctgggcctgccgccgattggcaaaaccgaaaaaaccggcaaacgtagcaccagcgcggcggttctggaagcgctgcgtgaagcgcatccgattgtggaaaaaatcctgcaatatcgtgagctggccaaactgaaaggcacctatattgatccgctgccggccctggtgcatccgaaaaccggccgtctgcatacccgttttaaccagaccgcgaccgcgaccggtcgtctgagcagcagcgatccgaacctgcaaaacattccggtgcgtaccccgctgggccagcgtattcgtcgtgcgtttgtggccgaagaaggctatctgctggttgcgctggattaagccagattgagctgcgtgttctggcccacctgagcggcgatgaaaatctgattcaggtgtttcaggaaggccgcgatattcatacccagaccgcgagctggatgtttggcctgccggccgaagcgatcgatccgctgatgcgtcgtgcggcgaaaaccattaactttggcgtgctgtatggcatgagcgcgcatcgcctgagccaggagctgagcattccgtacgaagaagcggtggcgtttattgatcgttatttccagagctacccgaaagtgaaagcgtggattgaacgtaccctggaagaaggccgtcagcgcggctatgtggaaaccctgtttggccgtcgtcgttatgtgccggatctgaacgcgcgtgtgaaaagcgttcgtgaagcggcggaacgtatggcgtttaacatgccggttcagggcaccgcggcggatctgatgaaactggcaatggtgcgtctgtttccgcgtctgccggaagtgggtgcgcgtatgctgctgcaagtgcatgatgagctgctgctggaagccccgaaagaacgtgcggaagcggcggcagccctggccaaagaagtgatggaggcgtttggccgctggccgtgccgctggaagttgaagtgggcattggtgaagattggctgagcgccaaatgctaa

SEQ ID NO: 6 - protein, 834 aa, Thermus eggertssonii
MGRGMLPLFEPKGRVLLVDGHHLAYRNFFALKGLTTSRGEPVQGVYGFAKSLLKALKEDGDVVIVVFDAKAPFFRHEAYEAYKAGRAPTPEDFPRQLALIKELVDLLGLERLEVPGFEADDVLATLAKQAEREGYEVRILTADRDLFQLLSDRIAVLHPEGHLITPGWLWERYGLKPEQWVDFRALAGDPSDNIPGVKGIGEKTALKLLKEWGSLENLLKNLDHVKPPSVREKILAHLDDLRLSQELSRVRTDLPLKVDFKKRREPDREGLKAFLERLEFGSLLHEFGLLESPLPAEEAPWPPPEGAFLGYRLSRPEPMWAELLALAASAKGRVYRAEEPYGALRGLKEVRGLLAKDLAVLALREGLDLPPTDDPMLLAYLLDPSNTTPEGVARRYGGEWTEEAGERAVLSERLYENLLGRLRGEEKLLWLYEEVEKPLSRVLAHMEATGVRLDVAYLKALSLEVAEEMRRLEEEVFRLAGHPFNLNSRDQLERVLFDELGLPPIGKTEKTGKRSTSAAVLEALREAHPIVEKILQYRELAKLKGTYIDPLPALVHPKTGRLHTRFNQTATATGRLSSSDPNLQNIPVRTPLGQRIRRAFVAEEGYLLVALDYSQIELRVLAHLSGDENLIQVFQEGRDIHTQTASWMFGLPAEAIDPLMRRAAKTINFGVLYGMSAHRLSQELSIPYEEAVAFIDRYFQSYPKVKAWIERTLEEGRQRGYVETLFGRRRYVPDLNARVKSVREAAERMAFNMPVQGTAADLMKLAMVRLFPRLPEVGARMLLQVHDELLLEAPKERAEAAAALAKEVMEGVWPLAVPLEVEVGIGEDWLSAKC

SEQ ID NO: 7 - DNA, 2503 bp, Thermus eggertssonii
atgggccgtggtatgctgccgctgtttgaaccgaaaggccgtgtgctgctggttgatggccaccatctggcctatcgtaacttttttgcgctgaaaggcctgaccacgagccgtggtgaaccggtgcagggcgtgtatggctttgcgaaaagcctgctgaaagcgctgaaagaggatggcgacgttgttattgtggtgtttgatgcgaaagcgccgttttttcgtcatgaagcgtacgaagcgtataaagcgggccgtgcgccgaccccggaagattttccgcgtcagctggccctgattaaagagctggttgatctgctgggcctggaacgtctggaagtgccgggctttgaagcggatgatgtgctggccaccctggccaaacaggcggaacgtgaaggctatgaagtgcgtattctgaccgcggatcgtgacctgtttcagctgctgagcgatcgtattgcggtgctgcatccggaaggccatctgattacgccgggctggctgtgggaacgttatggcctgaaaccggaacagtgggtggattttcgtgcgctggccggcgatccgagcgataacattccgggcgtgaaaggcattggcgaaaaaaccgcgctgaaactgctgaaagaatggggcagcctggaaaatctgctgaaaaacctggatcatgtgaaaccgccgagcgtgcgtgaaaaaattctggcccatctggatgatctgcgtctgtctcaggagctgtctcgcgttcgtaccgatctgccgctgaaagtggattttaaaaaacgtcgtgaaccggatcgtgaaggcctgaaagcgtttctggaacgcctggaatttggcagcctgctgcatgaatttggcctgctggaaagcccgctgccggcggaagaggcgccgtggccgccaccggaaggtgcgtttctgggctatcgtctgagccgtccggaaccgatgtgggcggagctgctggccctggccgcgagcgcgaaaggtcgtgtgtatcgtgcggaagaaccgtatggcgcgctgcgtggcctgaaagaagtgcgcggcctgctggctaaagacctggccgtgctggccctgcgtgaaggtctggatctgccgccgaccgatgatccgatgctgctggcctatctgctggacccgagcaacaccaccccggaaggtgtggcgcgtcgttatggcggcgaatggaccgaagaagcgggcgaacgcgcggttctgagcgaacgtctgtatgaaaacctgctgggccgtctgcgtggcgaagaaaaactgctgtggctgtatgaagaagtggaaaaaccgctgagccgtgtgctggcccatatggaagcgaccggcgtgcgtctggatgtggcgtatctgaaagccctgagcctggaagtggcggaagaaatgcgtcgtctggaagaagaagtgtttcgtctggccggccatccgtttaacctgaacagccgtgatcagctggaacgtgtgctgtttgatgagctgggcctgccgccgattggcaaaaccgaaaaaaccggcaaacgtagcaccagcgcggcggttctggaagcgctgcgtgaagcgcatccgattgtggaaaaaatcctgcaatatcgtgagctggccaaactgaaaggcacctatattgatccgctgccggccctggtgcatccgaaaaccggccgtctgcatacccgttttaaccagaccgcgaccgcgaccggtcgtctgagcagcagcgatccgaacctgcaaaacattccggtgcgtaccccgctgggccagcgtattcgtcgtgcgtttgtggccgaagaaggctatctgctggttgcgctggattaagccagattgagctgcgtgttctggcccacctgagcggcgatgaaaatctgattcaggtgtttcaggaaggccgcgatattcatacccagaccgcgagctggatgtttggcctgccggccgaagcgatcgatccgctgatgcgtcgtgcggcgaaaaccattaactttggcgtgctgtatggcatgagcgcgcatcgcctgagccaggagctgagcattccgtacgaagaagcggtggcgtttattgatcgttatttccagagctacccgaaagtgaaagcgtggattgaacgtaccctggaagaaggccgtcagcgcggctatgtggaaaccctgtttggccgtcgtcgttatgtgccggatctgaacgcgcgtgtgaaaagcgttcgtgaagcggcggaacgtatggcgtttaacatgccggttcagggcaccgcggcggatctgatgaaactggcaatggtgcgtctgtttccgcgtctgccggaagtgggtgcgcgtatgctgctgcaagtgcatgatgagctgctgctggaagccccgaaagaacgtgcggaagcggcggcagccctggccaaagaagtgatggaggcgtttggccgctggccgtgccgctggaagttgaagtgggctgtggtgaagattggctgagcgccaaaggctaa

SEQ ID NO: 8 - protein, 834 aa, Thermus eggertssonii
MGRGMLPLFEPKGRVLLVDGHHLAYRNFFALKGLTTSRGEPVQGVYGFAKSLLKALKEDGDVVIVVFDAKAPFFRHEAYEAYKAGRAPTPEDFPRQLALIKELVDLLGLERLEVPGFEADDVLATLAKQAEREGYEVRILTADRDLFQLLSDRIAVLHPEGHLITPGWLWERYGLKPEQWVDFRALAGDPSDNIPGVKGIGEKTALKLLKEWGSLENLLKNLDHVKPPSVREKILAHLDDLRLSQELSRVRTDLPLKVDFKKRREPDREGLKAFLERLEFGSLLHEFGLLESPLPAEEAPWPPPEGAFLGYRLSRPEPMWAELLALAASAKGRVYRAEEPYGALRGLKEVRGLLAKDLAVLALREGLDLPPTDDPMLLAYLLDPSNTTPEGVARRYGGEWTEEAGERAVLSERLYENLLGRLRGEEKLLWLYEEVEKPLSRVLAHMEATGVRLDVAYLKALSLEVAEEMRRLEEEVFRLAGHPFNLNSRDQLERVLFDELGLPPIGKTEKTGKRSTSAAVLEALREAHPIVEKILQYRELAKLKGTYIDPLPALVHPKTGRLHTRFNQTATATGRLSSSDPNLQNIPVRTPLGQRIRRAFVAEEGYLLVALDYSQIELRVLAHLSGDENLIQVFQEGRDIHTQTASWMFGLPAEAIDPLMRRAAKTINFGVLYGMSAHRLSQELSIPYEEAVAFIDRYFQSYPKVKAWIERTLEEGRQRGYVETLFGRRRYVPDLNARVKSVREAAERMAFNMPVQGTAADLMKLAMVRLFPRLPEVGARMLLQVHDELLLEAPKERAEAAAALAKEVMEGVWPLAVPLEVEVGCGEDWLSAKG

SEQ ID NO: 9 - DNA, 690 bp, Escherichia coli
atggctaacgaattaacctggcatgacgtgctggctgaagagaagcagcaaccctattttcttaatacccttcagaccgtcgccagcgagcggcagtccggcgtcactatctacccaccacaaaaagatgtctttaacgcgttccgctttacagagttgggtgacgttaaagtggtgattctcggccaggatccttatcacggaccgggacaggcgcatggtctggcattttccgttcgtcccggcattgccattcctccgtcattattgaatatgtataaagagctggaaaatactattccgggcttcacccgccctaatcatggttatcttgaaagctgggcgcgtcagggcgttctgctactcaatactgtgttgacggtacgcgcaggtcaggcgcattcccacgccagcctcggctgggaaaccttcaccgataaagtgatcagcctgattaaccagcatcgcgaaggcgtggtgtttttgttgtggggatcgcatgcgcaaaagaaaggggcgattatagataagcaacgccatcatgtactgaaagcaccgcatccgtcgccgctttcggcgcatcgtggattctttggctgcaaccattttgtgctggcaaatcagtggctggaacaacgtggcgagacgccgattgactggatgccagtattaccggcagagagtgagtaa

SEQ ID NO: 10 - protein, 229 aa, Escherichia coli
MANELTWHDVLAEEKQQPYFLNTLQTVASERQSGVTIYPPQKDVFNAFRFTELGDVKVVILGQDPYHGPGQAHGLAFSVRPGIAIPPSLLNMYKELENTIPGFTRPNHGYLESWARQGVLLLNTVLTVRAGQAHSHASLGWETFTDKVISLINQHREGVVFLLWGSHAQKKGAIIDKQRHHVLKAPHPSPLSAHRGFFGCNHFVLANQWLEQRGETPIDWMPVLPAESE